(19) United States
(12) Patent Application Publication
(10) Pub. No.: US 2014/0256690 A1
(43) Pub. Date: Sep. 11, 2014

(54) CONTRACEPTIVE METHOD

(71) Applicants: Arkady RUBIN, Flemington, NJ (US); Maria FOEGH, Cresskill, NJ (US); Agis KYDONIEUS, Kendall Park, NJ (US); Thomas M. ROSSI, Stockton, NJ (US)

(72) Inventors: Arkady RUBIN, Flemington, NJ (US); Maria FOEGH, Cresskill, NJ (US); Agis KYDONIEUS, Kendall Park, NJ (US); Thomas M. ROSSI, Stockton, NJ (US)

(73) Assignee: AGILE THERAPEUTICS, INC., Princeton, NJ (US)

(21) Appl. No.: 14/201,153

(22) Filed: Mar. 7, 2014

Related U.S. Application Data
(60) Provisional application No. 61/775,331, filed on Mar. 8, 2013.

(51) Int. Cl. A61K 9/70; A61K 31/565; A61K 31/567

(52) U.S. Cl. CPC A61K 9/7023; A61K 31/567; A61K 31/565
USPC 514/170; 514/179

(57) ABSTRACT

A transdermal drug delivery device for reducing the risk of pregnancy in overweight women is disclosed. Methods of using the device are also disclosed. When used in accordance with the disclosed methods, the probability that the device will be effective in overweight women is approximately equal to or greater than the probability that the device would be effective in the case of a woman who is not overweight.

### FIELD OF THE INVENTION

This invention is in the field of transdermal delivery of gestagenic hormones to effect contraception in women.

### BACKGROUND OF THE INVENTION

A transdermal delivery device is an adhesive “patch” for application to the skin that is used to deliver a wide variety of pharmacologically and cosmetically active agents. Such patches can be used to deliver an agent transdermally, i.e., through the skin and into the bloodstream for systemic treatment or into or through the skin for local treatment. Such patches can also be used to administer topical treatments, including cosmetically active agents.

Such patches generally comprise, in addition to the active ingredient, i.e., the pharmaceutically or cosmetically active agent, an adhesive, a backing, and a release liner.

Various patches have been described for delivering gestagenic hormones, i.e., progestins, in order to effect contraception. Ortho Evra® norelgestromin/ethinyl estradiol transdermal system is commercially available in the US and elsewhere. Contraceptive patches are also described, e.g., in U.S. Pat. No. 7,045,145, U.S. Pat. No. 7,384,650, and US 20100255072.

It has been reported that overweight women have higher rates of pregnancy while on oral contraceptives than non-overweight women. See, e.g., Holt et al., Obstet Gynecol 2004, 99:820-7; Brunner et al., AEP 2006, 16(8):637-43. It has also been reported that Ortho Evra is less effective with overweight women than with non-overweight women. The Ortho Evra prescribing information reports that overweight women in a clinical study accounted for less than 3% of the study population but one-third of the pregnancies:
“With respect to weight, 5 of the 15 pregnancies reported with ORTHO EVRA® use were among women with a baseline body weight ≧198 lbs. (90 kg), which constituted <3% of the study population. The greater proportion of pregnancies among women at or above 198 lbs. was statistically significant and suggests that ORTHO EVRA® may be less effective in these women.
Health Care Professionals who consider ORTHO EVRA® for women at or above 198 lbs. should discuss the patient's individual needs in choosing the most appropriate contraceptive option.”

The prescribing information also states:
“PRECAUTIONS
1. Body Weight ≧198 lbs. (90 kg)
Results of clinical trials suggest that ORTHO EVRA may be less effective in women with body weight ≧198 lbs. (90 kg) than in women with lower body weights.”
   and
“GENERAL PRECAUTIONS 1. Weight ≧198 lbs. (90 kg) Clinical trials suggest that ORTHO EVRA may be less effective in women weighing 198 lbs. (90 kg) or more compared with its effectiveness in women with lower body weights. If you weigh 198 lbs. (90 kg) or more you should talk to your healthcare professional about which method of birth control may be best for you.”
   (198 lbs is approximately equal to a BMI of 33 Kg/m².)

### SUMMARY OF THE INVENTION

The invention relates generally to a method of using a transdermal hormone delivery system (“THDS”) comprising a progestin that comprises applying the THDS, i.e., the patch, to the skin of a woman who is overweight, e.g., excessively overweight, and/or who is a large woman.

In related aspects, the invention relates to a method of effecting contraception, i.e., reducing the risk of pregnancy, in an overweight and/or large woman that comprises successively applying a plurality of transdermal hormone delivery systems, each comprising a progestin, to the skin of a woman during a treatment period of at least three weeks duration.

In related aspects, the invention relates to a method of effecting contraception in an excessively overweight or large woman that comprises:

treating the woman by applying to the skin of the woman a transdermal contraceptive patch comprising levonorgestrel for wear during one or more treatment cycles, each treatment cycle comprising at least 3 consecutive one week dosing periods, whereby the probability that the patch will be effective is approximately equal to the probability that the patch would be effective in the case of a woman who is not excessively overweight.

In such illustrative embodiment, the woman may first be ascertained to belong to the subpopulation of women who are excessively overweight or large or to be selected from that subpopulation.

In related aspects, the invention relates to a method of improving contraceptive efficacy in a woman who is excessively overweight or large and who is receiving oral contraceptive treatment or transdermal contraceptive treatment, in particular, transdermal contraceptive treatment, e.g., OrthoEvra, said method comprising:

terminating the woman's current contraceptive treatment, in particular, the woman's current transdermal contraceptive treatment, and then
treating the woman by applying to the skin of the woman a transdermal contraceptive patch comprising levonorgestrel for wear during one or more treatment intervals, each treatment interval comprising at least 3 consecutive one week dosing periods,
whereby the probability that the patch will be effective is approximately equal to the probability that the patch would be effective in the case of a woman who is not excessively overweight.

In certain illustrative embodiments of the invention, an obese woman is treated for prevention of pregnancy employing a dosing regimen comprising a three week treatment interval followed by a one week rest period during which rest period the woman wears a transdermal device that provides low dose progestin and/or low dose estrogen during all or a part thereof. By low dose is meant a dose that delivers an amount of hormone(s) that may alleviate but does not prevent menstrual bleeding. In certain such illustrative embodiments, hormone is delivered for about ½ of the rest interval, i.e., two to four days.

In related aspects of the same invention, the invention comprises a method for conducting a pharmaceutical business, comprising: (a) manufacturing a transdermal patch or a kit comprising a transdermal patch, said patch comprising a progestin and an estrogen and (b) marketing to healthcare providers the benefits of using the patch or kit for effecting contraception in obese women.

In a similar vein, the invention comprises a method of marketing, i.e., promoting, a contraceptive transdermal patch that comprises providing instruction material to patients or physicians informing the patients or physicians that the patch is no less effective in obese women than it is in women who are not obese.

In particular illustrative embodiments, the patch comprises levonorgestrel and ethinyl estradiol and delivers these hormones in such doses as to result in exposure to levonorgestrel to be at least about 20-fold greater than exposure to ethinyl estradiol, e.g., the levonorgestrel:ethinyl estradiol blood concentration ratio is about 30 to about 50.

In related embodiments, the progestin is other than levonorgestrel and the ratio of exposure to the progestin to exposure to the estrogen is equivalent to a levonorgestrel:ethinyl estradiol ratio of at least 20, e.g., 30 to 50, based on the potency of the other progestin relative to the potency of levonorgestrel.

In some embodiments of the method of the invention, the patch comprises a backing layer and an adhesive polymer matrix affixed to the backing layer, wherein the adhesive matrix comprises:

a) a pressure sensitive adhesive polymer;
b) a humectant;
c) a skin permeation enhancer;
d) levonorgestrel.

Further illustrative embodiments of the invention include a method of using a THDS comprising a backing layer and an adhesive polymer matrix affixed to the backing layer and the adhesive matrix comprises:

a) a pressure sensitive adhesive polymer;
b) a humectant;
c) a skin permeation enhancer;
d) levonorgestrel;
said method comprising:
applying one THDS to the skin of the woman at the start of each week of a three week treatment interval;
providing a one week rest interval between each three week treatment interval;
wherein the woman is selected from the subpopulation of women who are excessively overweight or large and the probability that the THDS will be effective is approximately the same as the probability that the patch would be effective in the case of a woman who is not excessively overweight or large.

A further illustrative embodiment includes a method of improving contraceptive efficacy in a woman who is excessively overweight or large and who is receiving oral contraceptive treatment or transdermal contraceptive treatment, in particular, transdermal contraceptive treatment, said method comprising:

terminating the woman's current contraceptive treatment and then treating the woman by applying to the skin of the woman a transdermal contraceptive patch comprising levonorgestrel and ethinyl estradiol for wear during one or more treatment intervals, each treatment interval comprising at least 3 consecutive one week dosing periods, as described herein,
whereby the probability that the patch will be effective is approximately equal to the probability that the patch would be effective in the case of a woman who is not excessively overweight or large.

In some embodiments, the patch is a non-heat sealed transdermal drug delivery device and the adhesive matrix is an active ingredient layer comprising at least one volatile component and having a skin contacting surface and a non-skin contacting surface, said device further comprising:
prior to application, a release liner in direct contact with the skin-contacting surface of the AI layer, the perimeter of which extends beyond the perimeter of the AI layer in all directions; and
an overlay adjacent to the non-skin contacting surface of the AI layer, the perimeter of which extends beyond the perimeter of the AI layer in all directions; the overlay comprising a PSA layer, a polymeric intermediate layer, and an overlay covering;
the release liner and the PSA layer of the overlay are in contact with and adhered to each other around the perimeter of the AI layer to form a PSA seal between the overlay and the release liner;
the polymeric intermediate layer of the overlay prevents flow of the PSA of the PSA layer into the overlay covering; and
the solubility of the volatile component in the PSA of the PSA layer is less than the solubility of the volatile component in the PSA of the AI layer.

In all embodiments of the invention, the probability that the patch will be effective in the case of an excessively overweight or large woman is approximately equal to the probability that the patch would be effective in the case of a woman who is not excessively overweight or large.

In some of the embodiments described above and below, the probability that the transdermal contraceptive will be effective in an excessively overweight or large woman is even greater than the probability of efficacy in a woman who is not excessively overweight or who is not a large woman.

In all embodiments of the invention, the patch may also comprise an estrogen, one or more skin permeation enhancers, and other excipients such as but not limited to pressure sensitive adhesives, humectants, plasticizers, co-solvents, and antioxidants.

These and other embodiments of the invention that are described below are meant to be illustrative and not limiting.

### BRIEF DESCRIPTION OF THE FIGURES

FIG. 1. Exploded cross-section of an illustrative dermal delivery system for use in embodiments of the method of the invention.

FIG. 2. Mean serum ethinyl estradiol concentrations in healthy female volunteers follow application of the Patch on the buttock. Vertical arrow indicates time of patch removal.

FIG. 3. Mean serum levonorgestrel concentrations in healthy female volunteers follow application of the Patch on the buttock. Vertical arrow indicates time of patch removal.

FIG. 4. Mean Serum Concentration-Time Profiles of EE Following Once-Daily Administration of an Oral Contraceptive or Application of the Patch or Application of ORTHO EVRA® for 2 Cycles to the Buttock in Healthy Female Volunteers.

### DETAILED DESCRIPTION OF THE INVENTION

The present invention relates generally to transdermal delivery of a composition comprising a progestin for use in effecting contraception in excessively overweight women. There is variability in how one defines, “overweight,” and, “excessively overweight.” The National Heart, Lung, and Blood Institute of the US National Institutes of Health uses the following definitions:

| Category | Body Mass Index (Kg/m²) |
|---|---|
| Underweight | =<18.5 |
| Normal weight | 18.5 to 24.9 |
| Overweight | 25 to 29.9 |
| Obese | =>30 |

For purposes of describing an illustrative embodiment of this invention, “excessively overweight” will mean, “obese,” which will be used to describe a woman whose Body Mass Index (BMI) is equal to or greater than 30 Kg/m², ([(pounds/inches²)*703]=>30).

Body size, as measured by body weight, and BMI are not interchangeable. In other words, a high BMI person is not necessarily large, or unusually large, and a large person is not necessarily obese. Some people, of course, are both excessively overweight and large. For purposes of this specification, “large” is used to describe women who are =>90 Kg in body weight.

In the practice of this invention, a woman's weight status can be ascertained by any means, including, e.g., weighing the person on a weight scale, asking the person what her body weight, or approximate body weight, is, consulting the person's medical records, or visually observing the person.

In an illustrative embodiment of the invention, if a woman seeking contraception is excessively overweight, i.e., has a BMI=>30 and/or has a body weight of =>90 Kg, she can utilize a transdermal hormone delivery device, e.g., a patch, that delivers levonorgestrel and she can be confident, based on statistical analyses of populations of women who have used such patch, that the probability that the patch will be effective in preventing her from becoming pregnant will be approximately, if not exactly, or if not even greater than, what it would be were she in a different weight category.

In illustrative embodiments, the patch comprises a progestin, e.g., levonorgestrel, and an estrogen, such as estradiol or ethinyl estradiol. A treatment cycle typically comprises 4 weeks, having a 3 week treatment interval and a 1 week rest interval. During the treatment interval, a new patch is applied at the start of each week. During the rest interval, no patch is worn or a patch can be worn that is a placebo or that provides only low doses of a progestin or an estrogen or both.

In such illustrative embodiments, the mean steady state plasma concentration of ethinyl estradiol in a population of subjects (overweight and non-overweight) is about (e.g., +/−10%) 30 to about 50 pg/mL, e.g., 35 to 45 pg/mL, by the second week of a second treatment cycle, i.e., by the second week of a second treatment cycle, and during each week on patch during subsequent cycles, in patients with detectable levels of hormone (ethinyl estradiol or levonorgestrel) during patch wear. In certain such embodiments, the mean steady state plasma concentration of ethinyl estradiol in a population of subjects does not exceed about 70 pg/mL during any week of any treatment cycle. In certain such embodiments, the mean steady state plasma concentration of ethinyl estradiol in a population of subjects does not exceed about 60 pg/mL during any week of any treatment cycle. In certain such embodiments, the mean steady state plasma concentration of ethinyl estradiol does not exceed about 50 pg/mL during any week of any treatment cycle.

In illustrative embodiments, the mean steady state plasma concentration of levonorgestrel in a population of subjects (overweight and non-overweight) is about (e.g., +/−10%) 800 to about 2500 pg/mL, e.g., 900 to 2400 pg/mL, by the second week of a second treatment cycle, i.e., by the second week of a second treatment cycle, and during each week on patch during subsequent cycles, in patients with detectable levels of hormone (ethinyl estradiol or levonorgestrel) during patch wear.

In illustrative embodiments, the ratio of the mean steady state plasma concentration of levonorgestrel to the mean steady state plasma concentrations of ethinyl estradiol in a population of subjects (overweight and non-overweight) is about 30 to about 60.

The approved prescribing information for Ortho Evra (“Evra label”) provides data showing that the progestin (norelgestromin) to estrogen (ethinyl estradiol, EE) ratio ranges from 11.1 (Table 2 from Evra label) to 14.1 (Table 1 from Evra label). The same ratio may be reasonably assumed for obese and non-obese subjects. Although the prescribing information does not report data in obese women, the rates of decrease in norelgestromin and EE drug levels with increased body weight are comparable for other progestins and EE.

For the Patch that is specifically described below, the progestin:estrogen ratios range from 40.5 to 52.6 in non-obese subjects; and from 32.8 to 44.2 in obese subjects (Example 2, Study 1). In subjects with BMI <32, the ratios ranged from 50.8 to 53.1 (Example 3—PK Study).

It is evident that the LNG:EE ratios are much higher for the Patch when compared to Ortho Evra. Taking the lowest and highest ratios from the data discussed above, the ratio of progestin:EE in Ortho Evra is on the order of 11:1 to 14:1 whereas the ratio of progestin:EE in the Patch is on the order of 33:1 to 53:1. In other words, the ratio of LNG:EE achieved with the Patch is approximately 3 to 5 times greater than the norelgestromin:EE ratio achieved with Ortho Evra. In addition, the potency of levonorgestrel is greater than that of norelgestromin. Thus, the progestin:EE ratio, in terms of potency equivalence to levonorgestrel, is more than 3 to 5 times, e.g., about 12 times, greater for the Patch than for Ortho Evra.

As discussed above, the Ortho Evra prescribing information, and other references, indicate that the pregnancy rates for obese women receiving treatment with the Ortho Evra patch are greater (about 8× greater) than for non-obese women. However, in clinical studies of the Patch, described below, pregnancy rates were the same in obese and in non-obese women. Without intending to be bound to a particular mechanism, the lower progestin:estrogen exposure ratio achieved with Ortho Evra may explain higher pregnancy rates in overweight subjects.

Thus in an illustrative embodiment of the invention, the concentration of levonorgestrel in the blood of a patient is typically at least about 20-fold, e.g., 30- to 50-fold greater than the concentration of ethinyl estradiol. In the case of progestins and/or estrogens other than levonorgestrel and estradiol, the ratio of blood concentrations are equivalent, relative to the potency of the different progestin or estrogen.

In accordance with the practice of this invention, the contraceptive efficacy of a THDS, based on statistical analysis of a large population of women, is the same, or substantially the same, in excessively overweight women as it is in women who are not excessively overweight. For example, the Pearl Index (pregnancies per 100 women-years of use) in excessively overweight women and in non-excessively overweight women can both be in the range of 1 to 6 (95% confidence interval), e.g., 2.5 to 4.0, 3.0 to 3.5, or, 3.1 to 3.3.

In certain illustrative embodiments, the method of the invention employs a passive transdermal delivery device, e.g., a patch that comprises a polymeric matrix or a patch that comprises a reservoir with an optional rate controlling membrane. Alternatively, such device can be an active device such as an iontophoretic device.

An aspect of this invention is commercializing, i.e., marketing or promoting, a contraceptive THDS, e.g., a patch, by informing prescribers, insurers, and/or patients that the THDS is no less effective in obese women as it is in women of normal or light weight. A way to inform prescribers, insurers, or patients is to utilize instructional material comprising this information in connection with the marketing or sale of such THDS. Such instructional material may include such prescribing information as may be approved by a regulatory agency. In an illustrative such embodiment, the invention comprises a business method comprising the step of providing to a consumer a contraceptive transdermal device, or a kit comprising multiple such devices with or without a rest interval device, and prescribing information wherein said prescribing information instructs or directs said device to be administered to an obese woman to prevent contraception and that said device has been found to be no less effective in preventing pregnancies in obese women than in non-obese women. In the Unite States and in many, perhaps most, other countries, such information cannot legally be provided unless it has been substantiated in clinical trials. So, in related aspects, this invention comprising conducting at least one clinical trial with a THDS having as an objective of the trial comparing contraceptive efficacy in obese vs. non-obese women. The results of such trials can form a part of the dossier that is submitted to a regulatory agency, such as the US Food and Drug Administration, when seeking approval to market the THDS or when seeking to amend the conditions of approval, i.e., the “label.”

Useful THDS designs include those used for transdermal delivery of active pharmaceutical ingredients including but not limited to such devices for delivery of progestins and estrogens. Such device will comprise an active ingredient composition comprising a progestin and a carrier, e.g., a liquid carrier or a polymeric matrix.

Additional excipients, e.g., a humectant, a skin permeation enhancer, an antioxidant, etc., may also be included in the active ingredient composition.

An illustrative device that employs a polymeric matrix may comprise, as the active ingredient composition, e.g.:

a) a pressure sensitive adhesive polymer (“PSA”);
b) a progestin;
c) an estrogen;
d) a humectant;
e) a skin permeation enhancer; and
f) an antioxidant.

Below is a description of a transdermal hormone delivery system (THDS) that employs such active ingredient composition and that shows good adhesion. A THDS substantially as described below had excellent adhesion (over 90% of the surface area adhered for 7 days) in excessively overweight women and women not excessively overweight: BMI >35: 91.2%, BMI >=30<=35: 93.1%, and BMI <30: 93.3%. On a body weight basis, excellent adhesion (over 90% of the surface area adhered for 7 days) was observed in 93.3% of women below 90 Kg and in 92.1% of women above 90 Kg.

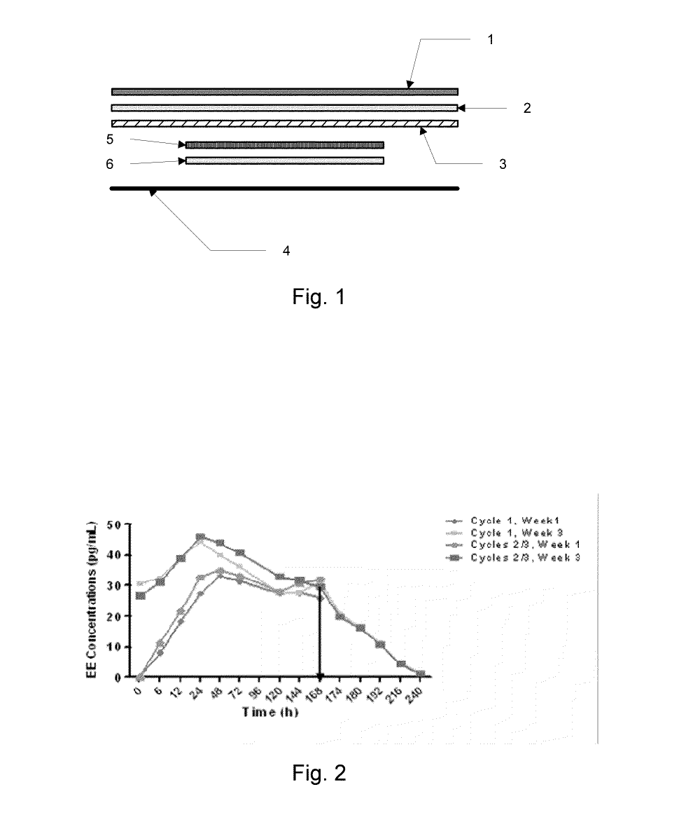

With reference to FIG. 1, this illustrative device for use in the practice of the invention comprises 4 layers. One is the active ingredient composition, i.e., the AI layer or AI patch (6). The second is a release liner (4). The third is an internal backing layer (5). The fourth is an overlay, which in this illustrative device, itself comprises three component layers (1,2,3), referred to herein below as, respectively, a PSA layer (3), an intermediate layer (2), and an overlay covering or overlay coating (1). The overlay can also be described as comprising, in this illustrative embodiment, a PSA layer (3) and an overlay covering (1 and 2). In any event, one feature of this embodiment is formation of a seal between the PSA layer (3) of the overlay (1,2,3) and the release liner (4).

The AI Layer

Layer 6 comprises the AI in a PSA matrix. In an illustrative embodiment of the invention, Layer 6 comprises the AI and a volatile skin permeation enhancer. The volatile component, however, can also be, for example, a solvent or carrier. Illustrative formulations of transdermal hormone compositions useful in delivery devices of the present invention are described, for example, in U.S. Pat. No. 7,045,145, U.S. Pat. No. 7,384,650, U.S. Pat. No. 8,246,978, and U.S. 20100292660.

In this illustrative embodiment, the AI is an active pharmaceutical ingredient (API) that is a progestin, e.g., levonorgestrel, and an estrogen, e.g., ethinyl estradiol or 17-13 estradiol, dispersed in an adhesive polymer matrix. In another device, the API is limited only to a progestin.

An illustrative Layer 6 is prepared as described in Example 1, below. This example describes formulations that use a combination of skin permeation enhancers, including an organic solvent such as DMSO and a lower (C1-C4) alkyl ester of lactic acid such as ethyl lactate, both of which are volatile components and are examples of volatile components that may be included in a transdermal drug delivery device of the invention. By “volatile,” is meant that the agent has a vapor pressure above 0.1 mm Hg at 20° C. Other illustrative volatile components useful in the present invention are known to those skilled in the art and include other volatile organic solvents, for example, sulfoxides such as decyl methyl sulfoxide; alcohols such as ethanol, propanols, hexanols, and benzyl alcohol, fatty acids such as valeric acid, isovaleric acid, isopropyl butyrate, ethyl acetate, and butyl acetate; polyols such as butanediol and ethylene glycol; amides such as dimethylacetamide, diethyl toluamide, dimethylformamide, pyrrolidone, and methyl pyrrolidone; terpenes such as limonene, pinene, terpinone, mentone, eucalyptus, and menthol; alkanes such as hexane and heptane, and organic acids such as citric acid.

Skin permeation enhancers and solvents additional to DMSO and similar organic solvents include but are not limited to those described in Example 1.

The following description relates to an illustrative formulation of Layer 6 for delivery of a contraceptive hormone(s), said layer, or patch, comprising a progestin and an estrogen, skin permeation enhancers, and a PSA matrix comprising an adhesive polymer and a humectant/plasticizer.

Skin Permeation Enhancers:

Drug molecules released from a transdermal delivery system must be capable of penetrating each layer of skin. In order to increase the rate of permeation of drug molecules, a transdermal drug delivery system, desirably, is able to increase the permeability of the outermost layer of skin, the stratum corneum, which provides the most resistance to the penetration of molecules. In this regard, the present invention allows for a transdermal drug delivery system that employs one or more skin permeation enhancers in specific amounts.

A number of skin permeation enhancers can be used to improve passage of drugs through the skin and into the blood stream. These include, e.g., alcohols; alkanones; amides and other nitrogenous compounds; 1-substituted azacycloheptan-2-ones; bile salts; cholesterol; cyclodextrins and substituted cyclodextrins; ethers; saturated and unsaturated fatty acids; saturated and unsaturated fatty acid esters; saturated and unsaturated fatty alcohol esters; glycerides and monoglycerides; organic acids; methyl nicotinate; pentadecalactone; polyols and esters thereof; phospholipids; sulfoxides; surfactants; terpenes; and combinations thereof.

As specific examples, the following can be mentioned decanol, dodecanol, 2-hexyl decanol, 2-octyl dodecanol, oleyl alcohol, undecylenic acid, lauric acid, myristic acid and oleic acid, fatty alcohol ethoxylates, esters of fatty acids with methanol, ethanol or isopropanol, methyl laurate, ethyl oleate, isopropyl myristate and isopropyl palmitate, esters of fatty alcohols with acetic acid or lactic acid, lauryl lactate, oleyl acetate, 1,2-propylene glycol, glycerol, 1,3-butanediol, dipropylene glycol and polyethylene glycols.

A combination of skin permeation enhancing agents is preferably employed in the practice of the present invention for delivery of levonorgestrel and ethinyl estradiol (EE) or 17 beta-estradiol. The combination comprises a mixture of (1) a pharmaceutically acceptable organic solvent, such as dimethyl sulfoxide (DMSO), (2) a fatty (C8-C20) alcohol ester of a hydroxy acid, such as lauryl lactate, (3) a lower (C1-C4) alkyl ester of a hydroxy acid, e.g., ethyl lactate, and (4) a C6-C18 fatty acid, such as capric acid. In specific embodiments, the fatty alcohol ester of lactic acid is lauryl lactate and the lower alkyl ester of lactic acid is ethyl lactate. A medium- to long-chain fatty acid in the skin permeation enhancer formulation can be employed among the skin permeation enhancers.

Capric acid is preferred for use but other C6-C18 saturated or unsaturated fatty acids may be used, including but not limited to caproic acid, caprylic acid, lauric acid and myristic acid, to name a few.

These skin permeation enhancers can be present in amounts as described below. In certain embodiments, one or more of the skin permeation enhancers may be eliminated from the polymer matrix.

In a particular such embodiment, the pharmaceutically acceptable organic solvent is DMSO. Other organic solvents suitable for use in the present invention include, but are not limited to, C1-C8 branched or unbranched alcohols, such as ethanol, propanol, isopropanol, butanol, isobutanol, and the like, as well as azone (laurocapram: 1-dodecylhexahydro-2H-azepin-2-one) and methylsulfonylmethane, to name a few.

The fatty alcohol ester of a hydroxy acid can be a fatty alcohol ester of lactic acid, such as lauryl lactate. However, other hydroxy acids and fatty alcohols may be utilized. Alternative hydroxy acids include, but are not limited to, alpha-hydroxy acids such as glycolic acid, tartaric acid, citric acid, malic acid and mandelic acid, as well as the beta-hydroxy acid, salicylic acid. Alternative fatty alcohols include any C8-C20 saturated or unsaturated fatty alcohols, such as myristyl, palmityl or oleyl alcohols, to name a few.

The lower alkyl ester of hydroxy acid can also utilize lactic acid, and can be, e.g., ethyl lactate. However, other hydroxy acids, such as glycolic acid, tartaric acid, citric acid, malic acid, mandelic acid and salicylic acid, may also be utilized. In addition isopropylmyristic acid (IPM) may be used as a substitute for the lower alkyl ester of hydroxy acid.

The aforementioned combination of skin permeation enhancers may be used to enhance transdermal delivery of steroid hormones from any type of transdermal delivery device.

An adhesive polymer matrix-type system as described in detail herein is preferred for use; however, the enhancer combination may also be utilized in non-adhesive polymers, as well as in multi-layer or reservoir-type transdermal delivery systems, to name a few.

The skin permeation enhancer is typically present in a concentration of at least 1% or at least 2% by weight of the composition. It may be present in a concentration of up to 50% or up to 40% by weight of the composition. In certain embodiments, the skin permeation enhancer is present in a concentration based on weight of the composition (i.e., wt %) of 1 to 50% or 10 to 40% or 20 to 30% of the composition.

It may be possible to achieve sufficient permeation of certain progestins, e.g., desogrestrel, without addition of a skin permeation enhancer.

Hormones:

A transdermal drug delivery device utilizing the aforementioned skin permeation enhancers can be used to deliver many progestins. In one embodiment, a combination of a progestin and an estrogen is utilized to effect contraception.

Progestins:

Progestins useful in the practice of the present invention include desogestrel, dihydroprogesterone, drospirenone, ethynodiol acetate, ethynodiol diacetate, etogestrel, gestodene, gestogen, 17-hydrogesterone, hydroxyprogesterone caproate, 3-keto-desogestrel, levonorgestrel, medroxyprogesterone acetate, medroxyprogesterone diacetate, megestrol, megestrol acetate, normegesterol, norelgestromin, norethindrone (i.e., norethisterone), norethindrone acetate, norethynodrel, norgestimate, norgestrel, 19-nortestosterone, progesterone, nestorone, methoxyprogesterone, and dl-norgestrel or any combination of two or more of said progestins. Of particular interest, are levonorgestrel and norethindrone and norethindrone salts, e.g., norethindrone acetate. Levonorgestrel is a potent progestin on a weight-dose basis and may be selected for that or other reasons. The progestin is typically present in a concentration based on weight of the transdermal composition (i.e., wt %) of 0.1 to 3% or 0.2 to 2.0% or 0.5-1.5%. The concentration will vary based on various factors especially the skin permeation rate and the relative potency of the progestin.

Estrogens:

Estrogens useful in the practice of the present invention include, without limitation, ethinyl estradiol, 17-beta-estradiol, estradiol-3,17-diacetate; estradiol-3-acetate; estradiol 17-acetate; estradiol-3,17-divalerate; estradiol-3-valerate; estradiol-17-valerate; 3-mono-, 17-mono- and 3,17-dipivilate estradiol esters; 3-mono-, 17-mono- and 3,17-dipropionate estradiol esters; 3-mono-, 17-mono- and 3,17-dicyclo pentyl-propionate estradiol esters; and estrone. Of particular interest, is ethinyl estradiol. The estrogen is typically present in a concentration based on weight of the transdermal composition (i.e., wt %) of 0.1 to 3% or 0.2 to 2.0% or 0.5 to 1.5%, e.g., 0.5 to 1%.

Ethinyl estradiol and levonorgestrel are compatible and can be dispersed in the adhesive polymer formulation. Typically, a transdermal dosage unit designed for one-week therapy should deliver at least about 20 μg/day of levonorgestrel, e.g., about 50 to about 100 μg/day (or an equivalent effective amount of another progestin) and 10-50 μg/day of ethinyl estradiol (or an equivalent effective amount of another estrogen). Those respective amounts of progestin and estrogen are believed to be necessary to inhibit ovulation and to maintain normal female physiology and characteristics. In the present invention, the amount of levonorgestrel transdermally delivered is preferably 30 μg per day for more than one day to about one week with a 15 cm² transdermal delivery device.

Combinations of the above with estradiol itself (for example, a combination of estradiol and estradiol-17-valerate or further a combination of estradiol-17-valerate and estradiol-3,17-divalerate) can be used with beneficial results. For example, 15-80% of each compound based on the total weight of the estrogenic steroid component can be used to obtain the desired result. Other combinations can also be used to obtain desired absorption and levels of 17β-estradiol in the body of the subject being treated.

In certain illustrative embodiments of the invention, the amount of the estrogen delivered results in blood concentrations of no more than about 50 μg/day of ethinyl estradiol (or an equivalent effective amount of another estrogen).

It will be appreciated that the hormones may be employed not only in the form of the pure chemical compounds, but also in a mixture with other pharmaceuticals that may be transdermally applied or with other ingredients which are not incompatible with the desired objective as listed above. Thus, simple pharmacologically acceptable derivatives of the hormones such as ethers, esters, amides, acetals, salts and the like, if appropriate, may be used. In some cases, such derivatives may be preferred. The progestin compound and the estrogenic steroid are ordinarily dispersed or dissolved concurrently in fabricating the hormone-containing adhesive polymer matrix or they may be dispersed or dissolved separately.

Polymers Used as Active Patch Components:

The AI-containing layer can be a polymer matrix comprising the pharmaceutically or cosmetically active ingredient. The polymer can be a PSA to form a biologically acceptable adhesive polymer matrix, preferably capable of forming thin films or coatings through which the AI can pass at a controlled rate. Suitable polymers are biologically and pharmaceutically compatible, nonallergenic, insoluble in and compatible with body fluids or tissues with which the device is contacted. The use of water soluble polymers is generally less preferred since dissolution or erosion of the matrix would affect the release rate of the AI as well as the capability of the dosage unit to remain in place on the skin. So, in certain embodiments, the polymer is non-water soluble.

Useful PSAs include, e.g., polyacrylate PSAs (including, e.g., polyacrylate copolymers), rubber-based PSAs such as polyisobutylene PSAs (including, e.g., polyisobutylene copolymers, e.g., polyisobutylene/polybutene copolymers) and natural rubber PSAs, and silicone PSAs.

Preferably, polymers used to form a polymer matrix in the AI-containing layer have glass transition temperatures below room temperature. The polymers are preferably non-crystalline but may have some crystallinity if necessary for the development of other desired properties. Cross-linking monomeric units or sites can be incorporated into such polymers. For example, cross-linking monomers that can be incorporated into polyacrylate polymers include polymethacrylic esters of polyols such as butylene diacrylate and dimethacrylate, trimethylol propane trimethacrylate and the like. Other monomers that provide such sites include allyl acrylate, allyl methacrylate, diallyl maleate and the like.

A useful adhesive polymer formulation comprises a polyacrylate adhesive polymer of the general formula (I):

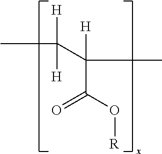

wherein X represents the number of repeating units sufficient to provide the desired properties in the adhesive polymer and R is H or a lower (C1-C10) alkyl, such as ethyl, butyl, 2-ethylhexyl, octyl, decyl and the like. More specifically, it is preferred that the adhesive polymer matrix comprises a polyacrylate adhesive copolymer having a 2-ethylhexyl acrylate monomer and approximately 50-60% w/w of vinyl acetate as a co-monomer. An example of a suitable polyacrylate adhesive copolymer for use in the present invention includes, but is not limited to, that sold under the tradename of Duro Tak® 87-4098 by National Starch and Chemical Co., Bridgewater, N.J., which comprises a certain percentage of vinyl acetate co-monomer.

Humectant/Plasticizer:

Preferably, a plasticizer/humectant is dispersed within the adhesive polymer formulation. Incorporation of a humectant in the formulation allows the dosage unit to absorb moisture from the surface of skin which in turn helps to reduce skin irritation and to prevent the adhesive polymer matrix of the delivery system from failing. The plasticizer/humectant may be a conventional plasticizer used in the pharmaceutical industry, for example, polyvinyl pyrrolidone (PVP). In particular, PVP/vinyl acetate (PVP/VA) co-polymers, such as those having a molecular weight of from about 50,000, are suitable for use in the present invention. The PVP/VA acts as both a plasticizer, acting to control the rigidity of the polymer matrix, as well as a humectant, acting to regulate moisture content of the matrix. The PVP/VA can be, for example, PVP/VA S-630 which is a 60:40 PVP:VA co-polymer that has a molecular weight of 51,000 and a glass transition temperature of 110° C. The amount of humectant/plasticizer is directly related to the duration of adhesion of the overlay. Preferably, the PVP/vinyl acetate is PVP/VA S-630 supplied by International Specialty Products, Inc. (ISP) of Wayne, N.J., wherein the PVP and the vinyl acetate are each present in approximately equal weight percent.

The shape of the device of the invention is not critical. For example, it can be circular, i.e., a disc, or it can be polygonal, e.g., rectangular, or elliptical. The surface area of the AI layer generally should not exceed about 60 cm² in area. Preferably, it will be about 5 to 50 cm², more preferably, about 8 to about 40 cm². Most preferably, the discs will be about 10 to about 20 cm². A disc of 15 cm² is preferred because of its relatively small size, yet being capable of dispersing high levels of hormones. Specific embodiments of the invention feature patches having an AI layer with a surface area of 10, 12.5, 15, 17.5 or 20 cm². However, other sizes may be utilized.

Other Components:

The active path matrix may comprise additional components. For example, it may comprise one or more antioxidants.

Anti-oxidants function to prevent or inhibit oxidation of other molecules by themselves becoming oxidized. In a polymeric matrix comprising both a progestin and an estrogen such as ethinyl estradiol, the ethinyl estradiol functions as an anti-oxidant and thereby helps to reduce oxidative degradation of the progestin. Employment of an additional anti-oxidant further reduces oxidative degradation. In a progestin-only composition, employment of an additional anti-oxidant can be even more important.

For example, certain polymers, in particular, polymers formed by free radical polymerization, have been found to contain residual levels of initiators that act as oxidizing agents in a polymeric matrix comprising a progestin, whereby the stability of the progestin is compromised. For example, polyacrylate adhesives matrices cause oxidation of a progestin, e.g., levonorgestrel.

PVP, which is commonly used in transdermal polymeric compositions, also contributes to oxidation of a progestin because of trace levels of residual initiators. Therefore, in transdermal compositions comprising PVP, or PVP/VA, and a progestin, addition of an anti-oxidant improves the stability of the progestin.

Certain permeation enhancers, e.g., DMSO, can also cause oxidation of a progestin, e.g., levonorgestrel.

Thus, one aspect of the invention features use of a polymeric matrix comprising the progestin, the anti-oxidant, the skin permeation enhancer and a pressure sensitive adhesive (“PSA”), wherein the PSA is a polyacrylate adhesive, e.g., a polyacrylate/vinyl acetate copolymer such as Duro Talc® 87-4098, and/or wherein the polymeric matrix comprises PVP or PVP/VA, and/or wherein the permeation enhancer comprises DMSO.

A number of compounds can act as anti-oxidants in a transdermal composition useful in the present invention. Among compounds known to act as anti-oxidants are: Vitamins A, C, D, and E, carotenoids, flavanoids, isoflavanoids, beta-carotene, butylated hydroxytoluene (“BHT”), butylated hydroxyanisole (BHA), glutathione, lycopene, gallic acid and esters thereof, salicylic acid and esters thereof, sulfites, alcohols, amines, amides, sulfoxides, surfactants, etc. Of particular interest are phenolic anti-oxidants, e.g., BHT, pentaerythritol tetrakis(3-(3,5-di-tert-butyl-4-hydroxyphenyl)propionate), e.g., Irganox 1010, and tris(2,4-di-tert-butylphenyl) phosphite, e.g., Irgafos 168, as well as sodium bisulfite, sodium sulfite, isopropyl gallate, Vitamin C and Vitamin E.

Phenolic anti-oxidants, like BHT, which are sometimes referred to as primary anti-oxidants, are particularly suitable. Larger phenolic anti-oxidants, e.g., molecular weight greater than 500 (e.g., tris(2,4-di-tert-butylphenyl)phosphite) or greater than 1000 (e.g., pentaerythritol tetrakis(3-(3,5-di-tert-butyl-4-hydroxyphenyl)propionate) may be utilized to advantage.

Anti-oxidants that would increase pH, e.g., sodium metabisulfite, are preferably avoided. BHT can be present, e.g., in a concentration based on the weight of the hormone of at least 10 wt % or at least 20 wt % or at least 30 wt % of the hormone. BHT can be present, e.g., in a concentration of up to 150 wt % or 200 wt % or 500 wt % of the hormone. In certain embodiments, BHT is present in a concentration based on weight of the hormone of 10 to 500%, 20 to 200%, or 50 to 150% of the hormone. Suitable concentrations of other anti-oxidants are readily ascertainable. For example, suitable concentrations of tris(2,4-di-tert-butylphenyl)phosphite, e.g., Irgafos 168, include concentrations that are similar to those of BHT, although lower or higher concentrations may also be employed; suitable concentrations of pentaerythritol tetrakis (3-(3,5-di-tert-butyl-4-hydroxyphenyl) propionate), e.g., Irganox 1010, include similar concentrations although lower or higher concentrations may be employed, e.g., concentrations that are up to about 10%, 20% or 30% higher.

The Internal Backing Layer

When the PSA comprises a polyacrylate matrix, as described above, the organic component can escape through the skin and non-skin contacting surface of the system. In order to minimize such escape through non-skin contacting surface, an internal backing layer can be employed. This layer, which inhibits absorption of components of the AI layer into the overlay, is illustrated as Layer 5 in FIG. 1.

Such internal backing layer can be made of any suitable material that is impermeable or substantially impermeable to the AI and to excipients of the adhesive polymer matrix. The internal backing layer serves as a protective cover for the AI layer and provides a support function. The backing layer can be formed so that it is essentially the same size as the hormone-containing adhesive polymer matrix or it can be of larger dimension so that it can extend beyond the edges of the AI-containing patch outwardly. The backing layer can be any appropriate thickness that will provide the desired protective and support functions. A suitable thickness is from about 10 to about 300 microns. More specifically, the thickness is less than about 150 microns, yet more specifically, it is less than about 100 microns, and most specifically, the thickness is less than about 50 microns.

Examples of materials suitable for making the internal backing layer are films of polypropylene, polyesters such as poly(ethylene terephthalate), metal foils, metal foil laminates of such suitable polymer films, and the like. Polyester films, such as Mylar® (DuPont Teijin) and Scotchpak® 9732 (3M Company), are particularly suitable for use in the present invention.

The Release Liner

The surface area of the release liner is greater than that of the AI layer. This can be seen in FIG. 1, where the diameter (in the case of a round device) or width and length (in the case of a polygonal device) of Layer 3 is greater than that of Layers 5 and 6, such that it extends beyond the AI layer in some or all directions.

The release liner is made of any material (1) that is impermeable or substantially impermeable to the components of the AI layer, (2) to which the PSA in the overlay will adhere, as discussed further hereinbelow, and (3) that is readily removable by peeling from the AI layer and overlay PSA just prior to applying to the skin.

The release liner can have the same dimensions as the overlay, discussed below, or it can extend totally or partially beyond the edge of the patch. In one illustrative embodiment, the release liner extends partially beyond the overlay so as to form “tabs” of release liner material that extend beyond the edges of the overlay for easy separation of the release liner from the rest of the system.

Preferably, it comprises a fluorinated or siliconized polyester film or another fluorinated or siliconized polymer such as a polyacrylonitrile copolymer, or a foil lined with a siliconized or fluorinated polymer. The release liner is preferably not polystyrene because it has been shown that polystyrene will absorb DMSO. A preferred material for the release liner when the layer 4a of the overlay comprises a PIB PSA is a Scotchpak® liner (3M Company), such as Scotchpak® 1022 or Scotchpak® 9744 fluorinated polyester release liners.

The Overlay

The overlay in this illustrative embodiment comprises a PSA in which the solubility of the volatile components is less, preferably significantly less, than the solubility of those same components in the AI matrix. So, e.g., when the volatile component is DMSO or ethyl lactate, a PIB PSA may be chosen. With reference to FIG. 1, the PIB PSA layer is Layer 3. Generally, such PIB PSA comprises a mix of a low to medium molecular weight and a high molecular weight PIB, a plasticizer such as polybutene, and a hydrocolloid such as a cross-linked polyvinylpyrrolidine. Useful PIBs include, e.g., Oppanol® PIBs (BASF), which have average molecular weights of between 40,000 and 4,000,000.

A useful PIB PSA comprises crospovidone such as Kollidon® CLM crospovidone (BASF) (e.g., 5-45 wt %, preferably 15-30 wt %, and more preferably 20-25 wt %); a low viscosity PIB such as Oppanol® B12 (molecular weight: 51000, viscosity at 150° C.: 150 Pascal-seconds) (e.g., 10-60 wt %, preferably 30-50 wt %); a high viscosity PIB such as Oppanol® B100 (viscosity: approximately 1100 Pascal-seconds) (e.g., 2-15 wt %, preferably 5-15 wt %); a polybutene such as Indopol® 1900 (Innovene LLC) (molecular weight: 2500, viscosity at 100° C.: 3900-4200 centistokes) (e.g., 10-60 wt %, preferably 20-40 wt %); and a mineral oil (0-20 wt %). For example, an illustrative formulation comprises about 20 wt % crospovidone, about 40 wt % of a low viscosity PIB, about 8 wt % of a high viscosity PIB and about 32 wt % of polybutene. (The term, “about,” as used in this specification, means plus or minus 10%. By “low viscosity” is meant less than about 300 Pascal-seconds and by “high viscosity” is meant more than about 800 Pascal-seconds, when the viscosity is measured at 150° C.) Cross-linking of the PVP is useful because such cross-linked polymers tend to be water-swellable but water insoluble. Such PIB PSA can provide good wear stability, e.g., attachment under normal living conditions for at least 7 days.

Other rubber-based polymers that can be used in place of PIB PSA in the overlay include silicone-based PSAs, such as BIO-PSA® (Dow Corning); copolymers and terpolymers of styrene/butadiene/styrene, styrene/isoprene/styrene, and styrene-ethylene/butylenes-styrene, such as Kraton D styrene/butadiene and Kraton G styrene-ethylene/butylene-styrene or styrene-ethylene/propylene-styrene. Isoprene rubbers, such as Kraton IR linear polyisoprene homopolymers, can also be used.

As shown in FIG. 1, and like the release liner, the overlay can extend beyond the perimeter of the AI layer in all directions, typically by a margin of about 0.1 to about 1.5 cm, more specifically about 0.3 to about 1.2 cm, and yet more specifically about 0.8 cm beyond the perimeter of the AI layer.

The overlay, if it comprises a PSA layer, improves adherence to the skin by supplementing the adhesion provided by the PSA in the AI layer, if present, or, in the case of an AI layer that does not comprise a PSA, it provides adherence to the skin.

In addition, in one illustrative embodiment of the invention, the overlay adheres to the release liner around the perimeter of both layers, thereby sealing in the components of the AI layer. By properly selecting the materials that comprise the overlay and the release liner, this seal between them prevents, or substantially prevents, escape of the volatile component in the AI layer but still allows the release liner to be peeled away easily by the user prior to topical application.

The seal is formed in situ by mechanically pressing together the edges of the overlay that extend beyond the perimeter of the AI layer and the edges of the release liner that extend beyond the perimeter of the AI layer. When the first overlay layer is a PIB PSA and the release liner is a fluorinated or siliconized polyester film, a suitable seal can be made by applying pressure. The amount of pressure required to form such seal is not critical. Finger pressure is adequate. Of course, in an illustrative embodiment of the invention, it is desirable that the seal can be broken by peeling the release liner from the rest of the system by hand just prior to application to the skin.

The seal between the overlay PSA and the release liner prevents, or substantially prevents, loss of the components of the AI layer through the seal between these two layers such as during storage of the system.

The overlay can also comprise a covering (1) that does not comprise a PSA, i.e., that comprises a non-PSA layer, such that the surface of the overlay that is exposed to fingers, clothing and ambient dirt or dust is non-tacky, is flexible or malleable so as to flex with skin and muscle movements, is of an unnoticeable or attractive color and texture, and permits moisture from the skin to pass through the device owing to its being porous or otherwise permeable to water.

Thus, it may be desirable to utilize a multi-layered overlay comprising a first layer of a PSA in which the volatile component is insoluble, covered with an intermediate layer and an overlay covering having the properties described above. Such illustrative overlay is illustrated in FIG. 1 as Layers 1, 2, and 3.

While a PIB PSA is useful for containing DMSO or ethyl lactate, or both, in the AI layer, the PIB PSA may flow through most overlay coverings having the properties described above. Such flow of the PIB PSA can cause the device to become tacky and discolored. Therefore, it may be desirable to use an overlay covering that itself comprises two layers, one of which is a polymeric layer interposed between the PIB PSA (an intermediate layer) and a backing layer. Such intermediate layer can be a polyacrylate PSA as described above, because such PSA will substantially prevent flow of the PIB PSA into and through the overlay covering but will substantially not itself migrate into or through the overlay covering.

Thus, in an illustrative embodiment of the invention, the AI layer comprises a polyacrylate matrix further comprising a humectant, e.g., PVP/VA, and skin permeation enhancers including DMSO, ethyl lactate, or both, or another one or more volatile organic solvents; the overlay is a laminate that comprises three layers: a PIB PSA layer (3, in FIG. 1); an intermediate layer that comprises a material that does not permit flow of the PIB PSA but that does permit passage of moisture (2, in FIG. 1); and an overlay covering (or backing layer) that is non-tacky, attractive, flexible, and moisture permeable (1, in FIG. 1).

Materials useful in the intermediate layer include, e.g., polyacrylates, polyurethanes, plasticized polyvinyl chlorides, and copolymers of polyethylene and ethyl vinyl acetate. Rubber-based polymers that are of very high molecular weight, e.g., at least about 150,000 Daltons can also be used, as can rubber-based polymers that can be crosslinked. Examples include the Kraton D styrene/butadiene, Kraton G styrene-ethylene/butylene-styrene or styrene-ethylene/propylene-styrene and Kraton IR linear polyisoprene homopolymers Butyl rubbers and silicone rubbers, which are cross-linkable, can also be used. The intermediate layer can comprise a PSA that binds the first overlay layer as well as the overlay covering. High molecular weight, cross-linked polymers are preferred. Preferably, such PSA is a polyacrylate such as is described above with reference to the AI layer.

Materials used in the overlay covering are not PSAs. They include, for example, a polyurethane film, foam or spun bonded structure, a polyolefin foam, a PVC foam or a woven or non-woven fabric. Illustrative wovens include KOB 051, 053 and 055 woven polyesters (Karl Otto Braun.) Illustrative non-woven fabrics include polyesters. An illustrative polyurethane material is CoTran™ 9700 melt-blown polyurethane nonwoven backing (3M), which can be colored in skin tones. Suitable materials are described, e.g., as backing layers in U.S. Pat. No. 6,660,295.

If the overlay covering is not porous, then it can be used without an intermediate layer. However, if the overlay covering is not porous, adhesion problems can result from a build up of moisture in the skin/PIB PSA interface. Use of a solid material, i.e., one that is not porous, but that is otherwise permeable to water, such as a thin, e.g., 1 mil (i.e., 0.001 inch), polyurethane film, can be used. However, a porous material such as a foam or fabric will, in general, better retain its shape and provide good adhesion.

Another aspect of an example of a device that can be used in the method of the invention pertains to use of a PIB PSA in an overlay for an AI layer that comprises a volatile solvent, especially, DMSO, because DMSO is poorly soluble in PIB PSAs. See Table 1, below, which compares the solubility of DMSO in a polyacrylate PSA (Duro Tak 87-4098, National Starch) and in a PIB PSA such as is described above.

TABLE 1
Saturation Solubilities (mg/g)

| PSA | DMSO | Ethyl Lactate | Lauryl Lactate |
|---|---|---|---|
| Duro-Tak 87-4098 | 8 | 150 | 1000 |
| PIB PSA | 0.01 | 0.03 | 785 |

These data indicate that DMSO and ethyl lactate, which are both volatile, cannot migrate into the PIB PSA because of saturation considerations. Of course, it will be understood that some amount of absorption into the overlay is acceptable and, indeed, unavoidable, at least under certain conditions. It is important, however, that the solubility of the volatile component in the AI containing layer be higher than, preferably substantially higher than, the solubility of the volatile component in the overlay PSA. References herein to a PSA that does not absorb volatile components must be understood in this context. In any event, the above data also indicate that the lauryl lactate, which is relatively not volatile, can flow into the PIB PSA by contact, which is why an internal backing layer is preferred in the transdermal drug delivery system of the invention.

Consistent with the above data, wear studies have shown that the PIB PSA retains its adhesiveness better than the polyacrylate PSA when stored in the presence of volatile enhancers owing to the reduced tendency of the volatile enhancers to migrate into the PIB PSA from an acrylic adhesive AI matrix, which migration would adversely affect the PIB PSA adhesiveness.

Overlay PIB and polyacrylate PSAs were tested in wear studies to determine their ability to adhere to skin for long periods of time. Table 2 shows that when absorption of excipients was minimized (25° C. exposure) the acrylic adhesive gave better results than the PIB. When absorption of excipients was allowed to proceed in a more rapid rate (40° C. exposure) the adhesion provided by the PIB PSA was better than that of the acrylic PSA. It is important to note that the adhesivity of the PIB PSA was the same when exposed to higher (40° C.) or lower (25° C.) conditions, for absorption of volatile excipients.

TABLE 2
Adhesivity of Integral Overlay/Active Patches

| Overlay | Equilibration | Equilibration | Adhesivity |
|---|---|---|---|
| Adhesive | Time | Temperature | (1) |
| Acrylic | 1 month | 25° C. | 16.2 |
| Acrylic | 1 month | 40° C. | 12.8 |
| PIB | 1 month | 25° C. | 15.1 |
| PIB | 1 month | 40° C. | 15.1 |

Note:
(1) These are relative values in which a higher number signifies better adhesion.

Another aspect of an example of a device that can be used in the method of the invention pertains to use of an overlay covering to cover the PIB PSA, which layer protects against contact with the PIB PSA and allows water vapor transmission. Another aspect of the invention pertains to use of a porous overlay covering and an intermediate layer that is permeable to moisture but that inhibits or prevents flow of the PIB PSA into and through the overlay covering.

The data in Table 3, below, illustrate that (1) use of a urethane overlay with or without a PIB PSA layer (PIB PSA is protecting the urethane on one side only) will result in absorption, and therefore loss, of volatile components such as are in the skin permeation enhancer composition illustrated in Examples 1 and 2 and (2) a polyester film does not absorb such components, even when coated with a PIB PSA such as when the internal backing layer is Mylar® and the overlay comprises a PIB PSA.

TABLE 3
Absorption of Enhancers by Patch Components (wt %)

| | | |
|---|---|---|
| 1. Polyurethane spun bonded | CoTran(TM) 9700 (3M) | 10.6 |
| nonwoven for overlay | | |
| 2. Same as 1 but coated with | Hi-Tack Nonwoven | 9.65 |
| acrylic adhesive | Medical Tape 9904 (3M) | |
| 3. Same as 2 but overcoated with 2 | | 7.6 |
| mm of PIB PSA (similar to Table 1) | | |
| Polyester internal backing layer | Mylar ® | 0.96 |
| Polyester coated with 2 mm PIB | Mylar ® | 1.12 |
| PSA(similar to Table 1) | | |

The data in Table 3 were obtained by placing in a metal dessicator the 4 enhancers in the same ratio as in the patch described in the Examples, below. The different components of the patch were placed in the same dessicator, making certain that the liquid enhancers (which were placed in a beaker on the bottom of the dessicator) were not in contact with the patch components. Therefore, any absorption of the enhancers into the patch components could only take place through vapor transfer. The dessicator was placed in a 40° C. oven and the absorption into the patch components was measured by weighing the samples and determining the weight gain after 3 months.

Polyester non-woven fabrics, e.g., KOB 053 and KOB 055, were also shown not to absorb the volatile components to a significant extent.

### EXAMPLES

The following examples are set forth to describe the invention in greater detail. They are intended to illustrate, not to limit, examples of devices that can be used in the practice of the invention and to provide data illustrating efficacy in overweight women.

### Example 1

### Fabrication of Transdermal Hormone Delivery System

Example 1 is a description of one of the ways to fabricate a THDS useful in the method of the invention. It will be appreciated that other ways can also be used. In this example, Part A illustrates preparation of Internal Backing/AI layer/Release Liner Laminate. Part B illustrates fabrication of a foam/acrylic PSA/PIB PSA overlay structure. Part C illustrates fabrication of an integrated device, or system, of the invention utilizing the laminates prepared in Parts A and B.

Part a. Fabrication of an Internal Backing/AI Layer/Release Liner Laminate:

After deaeration, an adhesive polymer composition comprising the AI and the volatile component(s) is applied to the backing layer material, and subsequently dried for a set time at a set temperature. In an alternative embodiment, the adhesive polymer matrix may be applied to a release liner instead of to the backing layer. Accordingly, reference herein to application of the adhesive polymer matrix to the backing layer will be understood to include this alternative embodiment. Application of the deaerated adhesive polymer matrix to the backing layer may be accomplished using commercially available laboratory coating/drying apparatus routinely used for this purpose. For instance, the Werner Mathis Model LTSV/LTH apparatus may be utilized, as well as other laboratory coating devices available from Werner Mathis AG (Zurich, Switzerland). Other suitable devices include, but are not limited to, instruments produced by Chemsultants, Inc. (Mentor, Ohio).

The thickness of the adhesive polymer solution applied to the backing layer, as well as the time and temperature of drying, are all process parameters that can be varied to achieve the final concentrations and ratios of hormones and permeation enhancing agents within the patch. For instance, it has been found that a change in the thickness of adhesive polymer matrix applied to the backing layer (e.g., from 300 to 800 μm) can result in an overall greater retention of volatile skin permeation enhancers when the other two process parameters, drying time and drying temperature, are held constant. In contrast, changing the drying time, e.g., from 5 to 25 minutes, or the drying temperature, e.g., from 40-100° C., can result in overall losses in retention of volatile skin permeation enhancers, to a greater or lesser degree depending on the enhancer.

Thus, it will be appreciated by those of skill in the art that, in addition to selection of appropriate amounts of starting materials in the adhesive polymer starting formulation, an appropriate combination of (1) initial thickness of the deaerated adhesive polymer solution spread on the backing layer, (2) drying time and (3) drying temperature may be selected to achieve the final composition of skin permeation enhancers and AIs in the device.

The dried adhesive polymer matrix is next laminated with a piece of release liner (such as Scotchpak® 1022 or 9744, 3M Co., St. Paul Minn.) (or backing layer, if the alternative embodiment is utilized), preferably of the same size to form a sheet of the transdermal hormone delivery systems.

Part B. Fabrication of a Non-Woven/Acrylic PSA/PIB PSA Overlay Laminate: The Fabrication of the Overlay is Performed in Two Steps.

In the first step, a PET-silicone coated release liner is unwound and a solution of an acrylic adhesive Duro-Tak 87-2852 is coated on the silicone side of the release liner. The web proceeds through heated ovens where the solvents are blown off and the release liner/solid acrylic PSA laminate is formed. The laminate proceeds toward a laminator unit where the 3M 9700 spunbonded non-woven is unwound and the acrylic PSA and the 3M 9700 go through the heated laminator rolls where a three layer laminate is formed (3 m 9700/acrylic PSA/silicone release liner).

In the second step a PET-silicone coated release liner is unwound and a solution of a PIB PSA is coated on the silicone side of the release liner. The web proceeds through heated rolls where the solvents are blown off and the release liner/solid PIB PSA laminate is formed. The laminate proceeds toward a laminator unit where the 3M 9700/acrylic PSA/silicone release liner laminate is positioned. The 3M 9700 laminate is unwound, its release liner is removed and discarded and the rest of the laminate proceeds toward the heated laminator rolls where it combines and gets laminated to the release liner/solid PIB PSA laminate to form the finished overlay composed of 3M 9700 spun bonded non-woven/acrylic PSA/PIB PSA/silicone release liner.

Part C. Fabrication of an Integrated Device of the Invention (Double Disc Conversion Process):

The conversion of a double disc, peripheral adhesive transdermal delivery device is fabricated on a die cutting-laminating piece of equipment typical for the industry. It has at least two payout stations, two die cutting stations, one lamination station, and three rewind stations. A roll of overlay laminate (Polyurethane, Polyacrylate PSA and PIB PSA) from Part B and a roll of release liner/active patch/internal backing layer laminate from Part A are mounted onto the payout spindles. The active patch laminate is threaded through a die cutting station where a partial or kiss cut is performed in the shape of the active patch through the internal backing and AI layer, and not through the release liner. The waste material around the patches is delaminated from the protective liner and wound onto a rewind spindle.

The overlay laminate is threaded through the conversion machine, the release liner is removed and the exposed overlay adhesive-urethane backing is laminated over the patch and onto the release liner from the active patch laminate. The resultant laminate with the active patch sandwiched between the overlay and the release liner is die cut in a shape larger than the active patch and collected for the next processing step. The resulting liner with holes cut out in the shape of the overlay-patch is wound on a rewind spindle.

Dermal delivery devices of the invention can optionally be packaged for distribution and sale to users. Standard packaging can be used or, if desired, packaging that exerts pressure on the in situ seal between the release liner and the PSA of the overlay can be employed. The purpose for such packaging is to keep the release liner and overlay in contact with each other and to minimize slippage or gapping that might occur, e.g., during transportation. Such packaging can comprise a closable packet (e.g., a clamshell packet that is hinged to open but that can be snapped close) that when closed fits snugly around the perimeter of the delivery device, thereby exerting a small amount of pressure on the in situ seal, but that is shaped so as not to squeeze the AI-containing patch.

### Example 2

### Contraceptive Efficacy Study

Two multicenter phase 3 clinical trials evaluated the safety and efficacy of a transdermal delivery device useful in the method of the invention (“Patch”) for pregnancy prevention. Both were comparative, open-labeled, two-arm studies with a treatment duration up to 13 cycles. Women with BMI 17-60 kg/m² were enrolled in both studies in a Patch arm and in a combination oral contraceptive (“COC”) (ethinyl estradiol/levonorgestrel) arm.

The Patch is the subject of a new drug approval application, NDA Number 204017.

Each Patch was prepared substantially as described in Example 1 and contained 2.60 mg levonorgestrel (LNG) and 2.30 mg ethinyl estradiol (EE). The Patch has an active hormone containing skin contact surface area of 15 cm² with a flexible overlay peripheral adhesive. The inactive components are polyisobutylene adhesive, woven polyester fabric, acrylic adhesives, crospovidone, copovidone, lauryl lactate, ethyl lactate, dimethyl sulfoxide, capric acid, polyester internal backing and polyester release liner.

### Example 2

### Study I

One of the studies was conducted in the US over one year (thirteen 28-day cycles) as a multicenter, open-label, two-arm Patch and COC safety and efficacy trial in sexually active women 17 to 40 years of age (mean age: 26.4 years). At cycle six the subjects in the COC group were continued in the Patch group. All subjects had drug level determinations done in cycles 2, 6, and 13. The racial demographic of the women randomized to the Patch group were: Caucasian (56.9%), African-American (22.9%), Hispanic (14.8%), Asian (3.4%), and Other (2.1%). Women of any body mass index (BMI) were included. The BMI range was 16-60 kg/m2 with 30% of the women being obese (BMI >=30 kg/m2) and half of obese women having a BMI of greater than 35 kg/m2. Among the women in the Patch group, 82.5% had not used hormonal contraception immediately prior to enrolling in this study and of those 68.4% were new users (no prior hormonal contraceptive use). Of treated women, 19.3% were lost to follow-up, 10.1% discontinued due to an adverse event, and 15.3% discontinued by withdrawing their consent. Similar pregnancy rates occurred in the Patch and COC groups, with a trend for lower pregnancy rate in the Patch group compared to COC group during cycles 1-6.

The pregnancy rate (Pearl Index [PI]) in women 17 to 35 years of age, excluding self-reported and proven non-compliant women, was 3.15 per 100 women-years of use (95% confidence interval 1.29-5.00) in non-obese subjects (BMI <30), 3.58 per 100 women-years of use (95% confidence interval 1.69-5.46) in non-obese subjects (baseline body weight <90 kg), 1.33 per 100 women-years of use (95% confidence interval 0.00-3.90) in obese subjects (baseline body weight >=90 kg), and 3.19 per 100 women-years of use in all subjects (95% confidence interval 1.58-4.80). This calculation was based on pregnancies that occurred after the onset of treatment and extending through the 14 days following the removal of the last patch. Cycles with no sexual activity were excluded as were cycles in which other birth control methods were used for reasons other than missed days of drug-taking. Cycles during which the subject missed one or more days of active drug taking and did not adhere to the procedures recommended for the missed days of drug taking as well as immediately following cycles were also excluded. Women with laboratory verified non-compliance were also excluded.

The attached Tables 14.2_{—}5.4.2 show mean and median EE and LNG levels during Cycles 2, 6, and 13.

Table 4 reports the levonorgestrel: ethinyl estradiol ratios based on the data in Tables 14.2-5.4.2.

TABLE 4
Contraceptive Efficacy Study I - Mean Drug Concentration Levels

| | | LNG | EE | LNG/EE |
|---|---|---|---|---|
| Study Period | BMI Category | (pg/mL) | (pg/mL) | Ratio |
| Cycle 2 | BMI < 30 | 1499 | 37.0 | 40.5 |
| | BMI >= 30 | 938 | 28.6 | 32.8 |
| | All BMIs | 1344 | 34.7 | 38.7 |
| Cycle 6 | BMI < 30 | 2198 | 45.5 | 48.3 |
| | BMI >= 30 | 1458 | 35.4 | 41.2 |
| | All BMIs | 1987 | 42.6 | 46.6 |
| Cycle 13 | BMI < 30 | 2034 | 38.7 | 52.6 |
| | BMI >= 30 | 1286 | 29.1 | 44.2 |
| | All BMIs | 1822 | 36.0 | 50.6 |

TABLE 14.2_5.4.2
Summary of EE and LNG Concentrations by Treatment Cycle and
BMI Category for Subjects with Detectable Concentrations
PK Population
BMI = <30 kg/m²

| | | | | AG200-15# | |
|---|---|---|---|---|---|
| | | | OC@ | AG200-15 | OC/AG200-15 |
| Parameter | Visit | Statistic | N = 232 | N = 669 | N = 113 |
| Ethinyl Estradiol (pg/mL) | Cycle 2 (Visit 3) | n | 201 | 590 | |
| | | Mean | 43.5 | 37.0 | |
| | | Median | 34.5 | 30.8 | |
| | | SD | 32.8 | 21.7 | |
| | | CV(%) | 75.2 | 58.8 | |
| | | Min, Max | (2.3, 174.0) | (2.9, 144.0) | |
| | Cycle 6 (Visit 5) | n | 139 | 387 | |
| | | Mean | 51.3 | 45.5 | |
| | | Median | 40.4 | 38.8 | |
| | | SD | 59.1 | 29.2 | |
| | | CV(%) | 115.3 | 64.3 | |
| | | Min, Max | (2.1, 618.0) | (2.3, 280.0) | |
| | Cycle 13 (Visit 7) | n | | 287 | 97 |
| | | Mean | | 38.7 | 41.8 |
| | | Median | | 31.6 | 36.5 |
| | | SD | | 27.6 | 26.1 |
| | | CV(%) | | 71.2 | 62.5 |
| | | Min, Max | | (2.2, 188.0) | (3.5, 120.0) |

SD = standard deviation, CV = coefficient of variation, BMI = body mass index.
@OC = Oral Contraceptive (100 mcg Levonorgestrel/20 mcg Ethinyl Estradiol).
#AG200-15: Subjects randomized to AG200-15. OC/AG200-15: Subjects randomized to OC who switched to AG200-15 after 6 cycles.

TABLE 14.2_5.4.2
Summary of EE and LNG Concentrations by Treatment Cycle and
BMI Category for Subjects with Detectable Concentrations
PK Population
BMI = >=30 kg/m²

| | | | | AG200-15# | |
|---|---|---|---|---|---|
| | | | OC@ | AG200-15 | OC/AG200-15 |
| Parameter | Visit | Statistic | N = 94 | N = 264 | N = 31 |
| Ethinyl Estradiol (pg/mL) | Cycle 2 (Visit 3) | n | 80 | 221 | |
| | | Mean | 31.6 | 28.6 | |
| | | Median | 25.9 | [symbol] | |
| | | SD | 21.7 | 20.7 | |
| | | CV(%) | 68.8 | 72.4 | |
| | | Min, Max | (5.9, 112.0) | (2.2, 215.0) | |
| | Cycle 6 (Visit 5) | n | 52 | 154 | |
| | | Mean | 34.1 | 35.4 | |
| | | Median | 24.4 | 30.6 | |
| | | SD | 27.6 | 25.0 | |
| | | CV(%) | 80.8 | 70.6 | |
| | | Min, Max | (2.2, 139.0) | (2.1, 140.0) | |
| | Cycle 13 (Visit 7) | n | | 111 | 28 |
| | | Mean | | 29.1 | 26.1 |
| | | Median | | 23.2 | 24.5 |
| | | SD | | 21.4 | 15.3 |
| | | CV(%) | | 73.6 | 58.5 |
| | | Min, Max | | (2.8, 118.0) | (3.1, 56.7) |

SD = standard deviation, CV = coefficient of variation, BMI = body mass index.
@OC = Oral Contraceptive (100 mcg Levonorgestrel/20 mcg Ethinyl Estradiol).
#AG200-15: Subjects randomized to AG200-15. OC/AG200-15: Subjects randomized to OC who switched to AG200-15 after 6 cycles. [symbol]
indicates data missing or illegible when filed

TABLE 14.2_5.4.2
Summary of EE and LNG Concentrations by Treatment Cycle and
BMI Category for Subjects with Detectable Concentrations
PK Population
BMI = <30 kg/m²

| | | | | AG200-15# | |
|---|---|---|---|---|---|
| | | | OC@ | AG200-15 | OC/AG200-15 |
| Parameter | Visit | Statistic | N = 232 | N = 569 | N = 113 |
| Levonorgestrel (pg/mL) | Cycle 2 (Visit 3) | n | 205 | 595 | |
| | | Mean | 2971 | 1499 | |
| | | Median | 2630 | 1280 | |
| | | SD | [symbol] | 993 | |
| | | CV(%) | 63.5 | 66.2 | |
| | | Min, Max | (79, 11500) | (98, 6530) | |
| | Cycle 6 (Visit 5) | n | 138 | 397 | |
| | | Mean | 3375 | 2198 | |
| | | Median | 3265 | 1880 | |
| | | SD | 1800 | 1511 | |
| | | CV(%) | 53.3 | 68.7 | |
| | | Min, Max | (346, 7630) | (58, 10700) | |
| | Cycle 13 (Visit 7) | n | | 289 | 102 |
| | | Mean | | 2034 | 2093 |
| | | Median | | 1580 | 1895 |
| | | SD | | 1549 | 1408 |
| | | CV(%) | | 76.1 | 67.3 |
| | | Min, Max | | (70, 11300) | (69, 7050) |

SD = standard deviation, CV = coefficient of variation, BMI = body mass index.
@OC = Oral Contraceptive (100 mcg Levonorgestrel/20 mcg Ethinyl Estradiol).
#AG200-15: Subjects randomized to AG200-15. OC/AG200-15: Subjects randomized to OC who switched to AG200-15 after 6 cycles. [symbol]
indicates data missing or illegible when filed

TABLE 14.2_5.4.2
Summary of EE and LNG Concentrations by Treatment Cycle and
BMI Category for Subjects with Detectable Concentrations
PK Population
BMI = >=30 kg/m²

| | | | | AG200-15# | |
|---|---|---|---|---|---|
| | | | OC@ | AG200-15 | OC/AG200-15 |
| Parameter | Visit | Statistic | N = 232 | N = 264 | N = 31 |
| Levonorgestrel (pg/mL) | Cycle 2 (Visit 3) | n | 85 | 228 | |
| | | Mean | 1790 | 938 | |
| | | Median | 1460 | 773 | |
| | | SD | 1201 | 758 | |
| | | CV(%) | 67.1 | 80.8 | |
| | | Min, Max | (107, 5780) | (57, 4220) | |
| | Cycle 6 (Visit 5) | n | 55 | 158 | |
| | | Mean | 2072 | 1458 | |
| | | Median | 1650 | 1180 | |
| | | SD | 1276 | 1125 | |
| | | CV(%) | 61.6 | 77.2 | |
| | | Min, Max | (155, 5110) | (72, 4860) | |
| | Cycle 13 (Visit 7) | n | | 114 | 28 |
| | | Mean | | 1286 | 1315 |
| | | Median | | 982 | 1230 |
| | | SD | | 1083 | 761 |
| | | CV(%) | | 84.2 | 57.9 |
| | | Min, Max | | (53, 6030) | (118, 4080) |

SD = standard deviation, CV = coefficient of variation, BMI = body mass index.
@OC = Oral Contraceptive (100 mcg Levonorgestrel/20 mcg Ethinyl Estradiol).
#AG200-15: Subjects randomized to AG200-15. OC/AG200-15: Subjects randomized to OC who switched to AG200-15 after 6 cycles.

### Example 2

### Study II

A smaller 407 subject comparator study was conducted in the US over 6 cycles as a multicenter, open label, two arm Patch and levonorgestrel-containing COC safety and efficacy trial in sexually active women 18-40 years of age (mean age: 25.7 years). All subjects had drug level determinations done at Cycles 3 and 6. Similar pregnancy rates occurred in the Patch and COC groups. The BMI of all study subjects was <32 Kg/m².

### Example 3

### Pharmacokinetics (PK)

Absorption

A PK study was conducted with 36 subjects for 3 cycles. All 36 subjects were on the Patch during Cycle 1. In cycle 2, 18 were on the Patch and 18 were on a combination oral contraceptive (“COC”) (Ortho-cyclen® norgestimate/ethinyl estradiol). In cycle 3, the Patch and COC arms were crossed over.

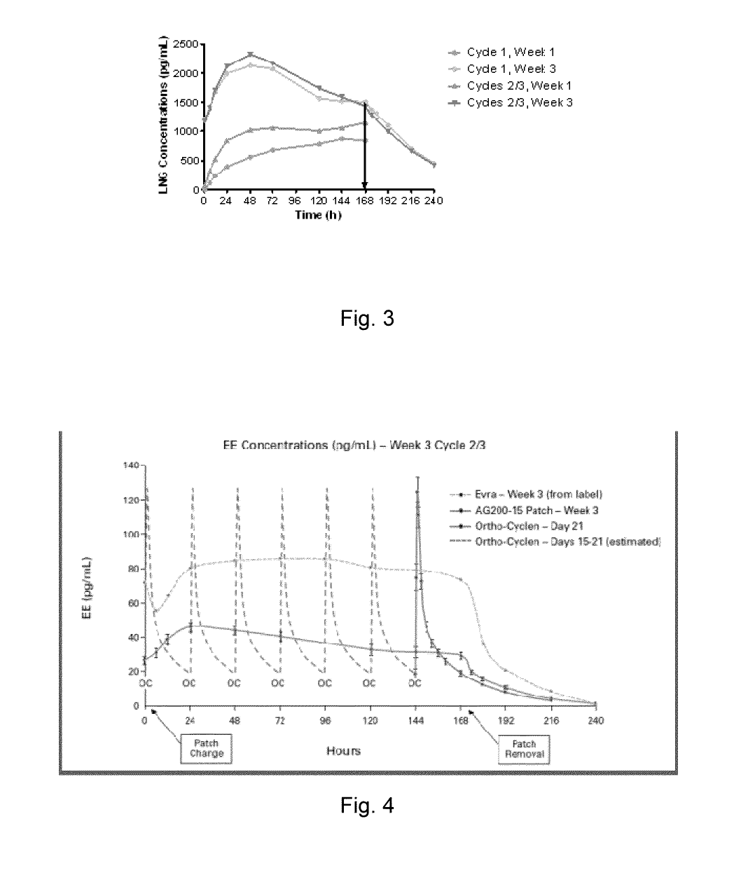

Following application of a patch as described above, both LNG and EE reach a plateau by 24 to 48 hours. Steady state is reached for EE by the 3rd week of Cycle 1 and by the 3rd week of cycle 2 for LNG (FIGS. 2 and 3). The mean steady state (Cₛₛ) concentrations are approximately 35 pg/mL for EE and 2200 pg/ml for LNG. EE exposure over two consecutive cycles of the patch therapy followed the pattern established for the COCs. When comparing the PK of LNG in 18 subjects over two consecutive cycles of patch wear, at Week 1 the maximum LNG concentration level (Cₘₐₓ) was about 75% higher in cycle 2 compared to cycle 1. During the third week of Patch wear, mean steady-state concentrations, as well as other PK parameters, were only about 10% higher in cycle 2 compared to cycle 1 (Table 5).

TABLE 5
Mean (% CV*) Pharmacokinetic Parameters of Levonorgestrel (LNG) and Ethinyl
Estradiol (EE) Following 2 Consecutive Cycles of “Patch” Wear on the Buttock

| | | Cycle 1 | | Cycle 1 | | Cycle 2 | | Cycle 2 | |
|---|---|---|---|---|---|---|---|---|---|
| | | Week 1 | | Week 3 | | Week 1 | | Week 3 | |
| Analyte | Parameter | (N = 18) | | (N = 18) | | (N = 18) | | (N = 18) | |
| LNG | Cₛₛ (pg/mL)^{a} | 842 | (41.2) | 2009 | (47.2) | 1389 | (46.5) | 2209 | (44.5) |
| | AUC₀₋₁₆₈ (ng · h/mL) | 120.0 | (39.1) | 339.0 | (41.1) | 207.0 | (44.1) | 378.0 | (43.8) |
| | t_{1/2} (h) | nc | | 38.2 | (22.7) | nc | | 40.5 | (15.4) |
| EE | Cₛₛ (pg/mL)a | 31.9 | (37.4) | 34.8 | (37.4) | 38.6 | (41.7) | 40.3 | (38.9) |
| | AUC₀₋₁₆₈ (pg · h/mL) | 5040 | (35.4) | 6210 | (34.2) | 6060 | (35.9) | 7120 | (36.6) |

| | | | | | | |
|---|---|---|---|---|---|---|
| t_{1/2} (h) | nc | 19.7 | (18.8) | nc | 20.5 | (18.2) |

aAverage concentration within the 48-168 h time-interval.
nc = not calculated

Exposure to ethinyl estradiol and levonorgestrel after treatment with the Patch was within the ranges reported for the low-dose LNG/EE oral contraceptive. The calculated daily doses of the Patch were equivalent to approximately 120 μg LNG and 30 μg EE.

Absorption of LNG and EE following application of the Patch to the buttock, abdomen and upper torso (excluding breast) was examined. While absorption from the abdomen was slightly lower than from other sites, exposure to EE and LNG for all anatomic sites was also within the ranges reported for the low-dose LNG/EE oral contraceptive and considered to be therapeutically equivalent.

Table 6 provides the summary of steady state pharmacokinetic parameters for ethinyl estradiol in women using the Patch compared with women using the COC.

TABLE 6
Mean (% CV) EE Steady State Pharmacokinetic Parameters Following
Application on the buttocks of the Patch and Once-Daily
Administration of an Oral Contraceptive (containing NGM
250 mcg/EE 35 mcg) in Healthy Female Volunteers

| | | |
|---|---|---|
| | Patch^{a} | Oral Contraceptive^{b} |
| Parameter | (N = 32) | (N = 32) |

| | | | | |
|---|---|---|---|---|
| Cₘₐₓ (pg/mL) | 51.3 | (33.7) | 131 | (34.7) |
| AUC₀₋₁₆₈ (pg · h/mL) | 6260 | (39.3) | 6970 | (32.2)^{d} |
| Cₛₛ (pg/mL) | 35.7 | (40.7)^{c} | 41.5 | (32.2)^{e} |

^{a}Cycles 2/3, Week 3
^{b}Cycles 2/3, Day 21
^{c}Average concentration within the 48-168 h time-interval.
^{d}Average weekly exposure, calculated as AUC₀₋₂₄ × 7
^{e}C_{avg}, calculated as (AUC₀₋₂₄)/24

The maximum EE concentration level (Cₘₐₓ) was approximately 60% lower for the Patch compared to COC. The treatment difference for Cₘₐₓ (p<0.0001) was highly statistically significant. Steady-state concentration level, Cₘₐₓ for the Patch is approximately 15% lower for the Patch when compared to the average concentration level for COC comparator, C_{avg}. The difference between the treatment groups was statistically significant (p=0.0167). Much lower maximum concentration levels and average concentrations were also reported for the first week of patch wear. Between-treatment differences were statistically significant for all EE PK parameters. Based on Cₛₛ the calculated daily dose of the Patch was equivalent to approximately 30 μg EE.

The Patch was designed to deliver EE and LNG over a seven-day period while oral contraceptives are administered on a daily basis. FIG. 4 presents mean pharmacokinetic (PK) profiles for EE following administration of the COC (EE 35 μg and norgestimate 250 μg) compared to the 7-day transdermal Patch (containing EE 2.50 mg/LNG 2.60 mg) during cycle 2 and 3 in 36 healthy female volunteers. Also included in FIG. 4 are PK data from the Ortho-Evra Prescribing Information (as approved by the US FDA).

The pharmacokinetic (PK) profile for the Patch is different from the PK profile for oral contraceptives in that it has lower peak concentrations. When comparing EE exposure (AUC) for the Patch and the contraceptive patch (Ortho Evra®) the EE exposure from the Patch is approximately 50% less than for Ortho Evra (FIG. 4; Ag200-15 Patch).

Data from this PK study also indicated a relatively high LNG:EE ratio in the blood, as shown in Table 7.

TABLE 7
PK Study. Mean Steady State Drug Concentration Levels

| | BMI | LNG | EE | LNG/EE |
|---|---|---|---|---|
| Study Period | Category | (pg/mL) | (pg/mL) | Ratio |
| Cycle 2 or 3 | BMI <= 32 | 1886 | 37.1 | 50.8 |
| (Week 3) | | | | |
| Two Consecutive | BMI <= 32 | 2253 | 42.4 | 53.1 |
| Cycles (Cycle 2, | | | | |
| Week 3 | | | | |

These data support the offered explanation for the greater efficacy of the Patch in obese women as compared to OrthoEvra.

The present invention is not limited to the embodiments described and exemplified above, but is capable of variation and modification within the scope of the appended claims. Published patent applications and patents referenced in this specification are incorporated herein by reference as though fully set forth.

1. A method of effecting contraception in an excessively overweight or large woman that comprises:
treating the woman by applying to the skin of the woman a transdermal contraceptive patch comprising levonorgestrel for wear during one or more treatment cycles, each treatment cycle comprising a treatment interval of at least 3 consecutive weeks,
whereby the probability that the patch will be effective is not less than approximately the probability that the patch would be effective in the case of a woman who is not excessively overweight or large.

2. The method of claim 1 wherein, prior to treatment, the woman is first ascertained to be excessively overweight or large.

3. The method of claim 1 wherein the patch further comprises an estrogen.

4. The method of claim 3 wherein the patch comprises an estrogen that is ethinyl estradiol (EE) and wherein the mean steady state concentrations of EE and LNG by the second week of a second consecutive treatment cycle are 30 to 50 pg/mL for EE and 800 to 2500 pg/mL for LNG.

5. The method of claim 3 wherein the patch comprises an estrogen that is ethinyl estradiol (EE) and wherein the mean steady state concentrations of EE and LNG by the second week of a second consecutive treatment cycle are approximately 35-45 pg/mL for EE and approximately 900 to 2400 pg/mL for LNG.

6. The method of claim 3 wherein the patch comprises an estrogen that is ethinyl estradiol (EE) and wherein the mean steady state concentrations of EE does not exceed 70 pg/mL during any week of any treatment cycle.

7. The method of claim 3 wherein the concentration of progestin in the blood of a patient is typically at least about 20-fold, e.g., 30- to 50-fold, greater than the concentration of the estrogen, on the basis of equivalent potency to levonorgestrel.

8. A method of effecting contraception in a woman that comprises applying to the skin of the woman transdermal hormone delivery systems (“THDS”), wherein
each THDS comprises a backing layer and an adhesive polymer matrix affixed to the backing layer and the adhesive matrix comprises:
a) a pressure sensitive adhesive polymer;
b) a humectant;
c) a skin permeation enhancer;
d) levonorgestrel;
one THDS is applied at the start of each week of a three week treatment interval;
each treatment interval is followed by a one week rest interval immediately prior to the start of each next treatment interval;
the woman is excessively overweight or large;
the probability that the THDS will be effective is the same as or greater than the probability that the patch would be effective in the case of a woman who is not excessively overweight or large.

9. The method of claim 8 wherein the adhesive polymer matrix further comprises ethinyl estradiol and wherein the concentration of progestin in the blood of a patient is typically at least about 20-fold, e.g., 30- to 50-fold, greater than the concentration of the estrogen, on the basis of equivalent potency to levonorgestrel.

10. The method of claim 9 in which the pressure sensitive adhesive polymer is a polyacrylate PSA, the humectant is PVP or PVP/VA, and the skin permeation enhancer comprises DMSO.

11. The method of claim 10 in which the skin permeation enhancer further comprises one or any combination of two or more of: a lower (C1-C4) alkyl ester of a hydroxy acid, a fatty (C8-C20) alcohol ester of lactic acid; a lower (C1-C4) alkyl ester of lactic acid; and a C6-C18 fatty acid.

12. The method of claim 11 in which the polyacrylate PSA is a polyacrylate copolymer.

13. The method of claim 12 wherein the polyacrylate copolymer comprises about 3 to about 60 wt % vinyl acetate.

14. The method of claim 13 wherein the humectant is PVP/VA and is about 60 wt % PVP and about 40 wt % VA.

15. The method of claim 12 wherein the fatty alcohol ester of lactic acid is lauryl lactate.

16. The method of claim 12 wherein the lower alkyl ester of lactic acid is ethyl lactate.

17. The method of claim 12 wherein the C6-C18 fatty acid is capric acid.

18. The method of claim 12 in which the skin permeation enhancer comprises lauryl lactate, ethyl lactate, and capric acid.

19. The method of claim 8 wherein the adhesive matrix further comprises an antioxidant.

20. A method of improving contraceptive efficacy in a woman who is excessively overweight or large and who is receiving transdermal contraceptive treatment, said method comprising:
terminating the woman's current contraceptive treatment and then
treating the woman by applying to the skin of the woman a transdermal contraceptive patch comprising levonorgestrel for wear during one or more treatment intervals, each treatment interval comprising at least 3 consecutive one week dosing periods,
whereby the probability that the patch will be effective is approximately equal to or greater than the probability that the patch would be effective in the case of a woman who is not excessively overweight.

21. The method of claim 20 wherein the woman's current contraceptive program is known to be less effective in women who are excessively overweight or large than in women who are not.

22. A method of effecting contraception in an excessively overweight or large woman that comprises:
treating the woman by applying to the skin of the woman a transdermal contraceptive patch comprising a progestin and an estroegn for wear during one or more treatment cycles, each treatment cycle comprising a treatment interval of at least 3 consecutive weeks,
wherein the concentration of progestin in the blood of a patient is typically at least about 20-fold, e.g., 30- to 50-fold, greater than the concentration of the estrogen, on the basis of equivalent potency to levonorgestrel and ethinyl estradiol,
whereby the probability that the patch will be effective is approximately the same as, or greater than, the probability that the patch would be effective in the case of a woman who is not excessively overweight or large.

23. The method of claim 22 wherein the progestin is levonorgestrel and the estrogen is ethinyl estradiol.

24. A method of effecting contraception in a woman that comprises applying to the skin of the woman transdermal hormone delivery systems (“THDS”), wherein
each THDS comprises a backing layer and an adhesive polymer matrix affixed to the backing layer and the adhesive matrix comprises:
a) a pressure sensitive adhesive polymer;
b) a humectant;
c) a skin permeation enhancer;
d) a progestin;
e) an estrogen
one THDS is applied at the start of each week of a three week treatment interval;
each treatment interval is followed by a one week rest interval immediately prior to the start of each next treatment interval;
the woman is excessively overweight or large;
the concentration of progestin in the blood of the woman is at least about 20-fold, e.g., 30- to 50-fold, greater than the concentration of the estrogen, on the basis of equivalent potency to levonorgestrel and ethinyl estradiol,
the probability that the THDS will be effective is the same as or greater than the probability that the patch would be effective in the case of a woman who is not excessively overweight.

25. The method of claim 24 wherein the progestin is levonorgestrel and the estrogen is ethinyl estradiol.

26. A method of marketing a contraceptive transdermal patch that comprises
providing instruction material to patients or physicians wherein the instruction material informs the patients or physicians that the patch is no less effective in obese women than it is in women who are not obese.
